(19) United States
(12) Patent Application Publication
(10) Pub. No.: US 2013/0110004 A1
(43) Pub. Date: May 2, 2013

(54) USER POSTURE DETECTION

(71) Applicants: Brian Paul McLane, The Woodlands, TX (US); Peter C. Ellis, Cupertino, CA (US); Michael C. Bartha, Houston, TX (US); Cynthia J. Purvis, The Woodlands, TX (US); John W. Frederick, Spring, TX (US)

(72) Inventors: Brian Paul McLane, The Woodlands, TX (US); Peter C. Ellis, Cupertino, CA (US); Michael C. Bartha, Houston, TX (US); Cynthia J. Purvis, The Woodlands, TX (US); John W. Frederick, Spring, TX (US)

(21) Appl. No.: 13/284,202

(22) Filed: Oct. 28, 2011

(51) Int. Cl. A61B 5/103

(52) U.S. Cl. 600/587

(57) ABSTRACT

Embodiments herein relate to detecting posture information. In an embodiment, a device detects the posture information related to a user's posture and stores the detected information. Further, the device receives input from the user about a region of the user's body experiencing pain and provides recommendations for a change to at least one aspect of the user's posture and the user's environment based on the stored information and the received input.

### BACKGROUND

A user may interact with a computing device over a long time period. During this time period, the user may experience pain or discomfort, such as from a musculoskeletal disorder, due to improper posture. For example, the user may experience neck or shoulder pain due to muscle strain.

Further, in a workplace, such user pain may result in a loss of productivity. The user learning proper posture may not be sufficient as the user may unknowingly revert back to an improper posture. Users and/or employers are challenged to find ways for the user to interact with the computing device over a long period of time without feeling pain or discomfort.

### BRIEF DESCRIPTION OF THE DRAWINGS

The following detailed description references the drawings, wherein:

FIG. 1 is an example block diagram of a computing device including instructions for detecting user posture;

FIG. 2 is an example block diagram of a device to detect user posture; and

FIG. 3 is an example flowchart of a method for detecting user posture.

### DETAILED DESCRIPTION

Specific details are given in the following description to provide a thorough understanding of embodiments. However, it will be understood by one of ordinary skill in the art that embodiments may be practiced without these specific details. For example, systems may be shown in block diagrams in order not to obscure embodiments in unnecessary detail. In other instances, well-known processes, structures and techniques may be shown without unnecessary detail in order to avoid obscuring embodiments.

Use of computing devices, such as desktop computers, has been associated with an increased number of cases of musculoskeletal disorders of the upper extremities (UEMSDs) and/or eye strain, due to improper posture over a long period of time by users. As a result, users may suffer from pain or discomfort such as neck or shoulder muscle strain and/or a loss of productivity in a workplace. Improving the user's environment, such as by including more ergonomic equipment, and/or learning of proper posture by the user may not be sufficient, as the user may unknowingly revert to the improper posture. For example, the user may round their back or crane their neck.

Monitoring software and/or hardware may be used to detect and notify the user of improper posture. Thus, the user may be reminded of the proper posture when they revert to the improper posture. However, constant notifications may be distracting to the user and result in lost productivity. Further, if the user is not experiencing pain or discomfort, the notifications may be unnecessary. For example, while the software may determine the user's posture to be improper, the user's posture may actually be proper and/or comfortable. Thus, the user may be able to maintain this posture without experiencing any pain or discomfort and/or the user may be more productive in this posture.

Accordingly, embodiments may provide a method and/or device that does not interrupt the user solely because a detected posture is determined to be improper. Instead, embodiments may allow the user to indicate when and where the user is feeling discomfort. For example, the user may be able to indicate when they begin to feel neck or should pain.

Further, embodiments may store a history or trend of the user's postures over time, which along with the user's indication of where they are experiencing discomfort, may allow embodiments to provide more in-depth and/or targeted recommendations about how the user should alter their posture in order to be more comfortable. For example, if the user states that they have neck pain, embodiments may analyze the user's stored history to determine that the user frequently engaged in a craned neck posture. Next, embodiments may suggest that the user alter a height of a display and/or a character zoom, to allow for easier viewing. Thus, embodiments may reduce musculoskeletal and visual discomfort as well as increase user wellness and productivity. In addition, embodiments may be relatively cost effective and easy to use and deploy, such as via a camera and user friendly software.

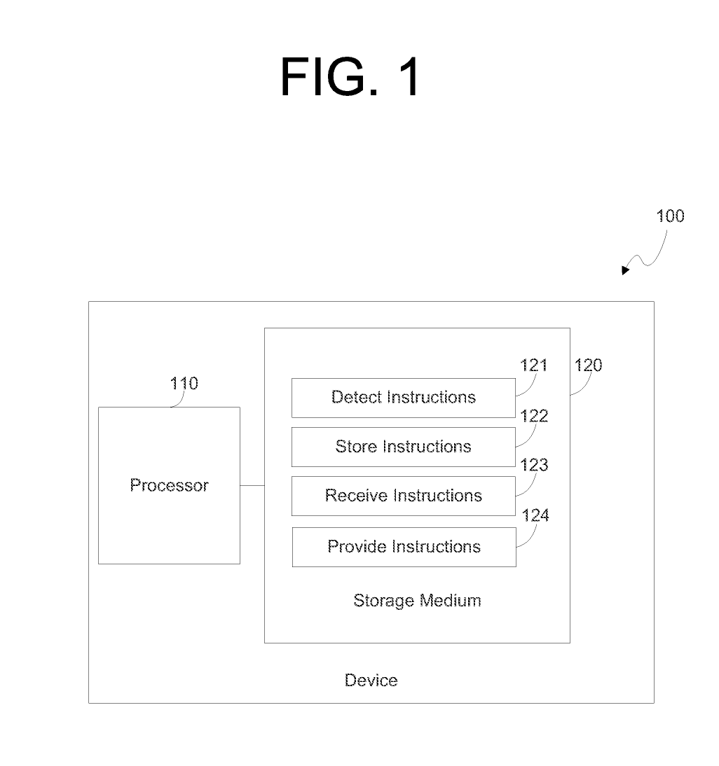

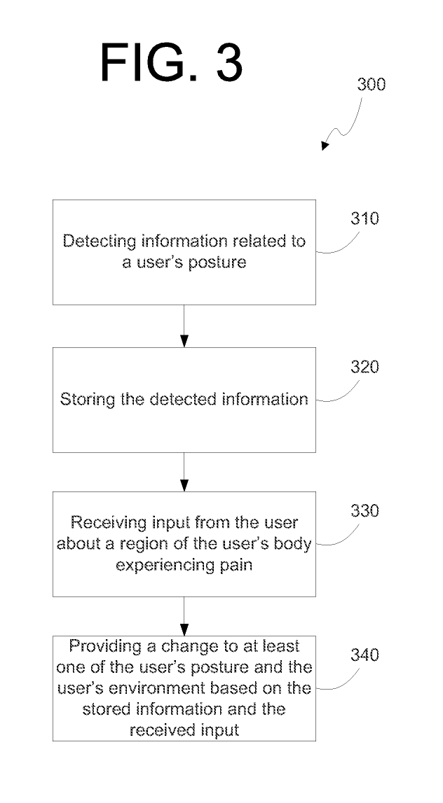

Referring now to the drawings, FIG. 1 is an example block diagram of a computing device 100 including instructions 121-124 for detecting user posture. In the embodiment of FIG. 1, the computing device 100 includes a processor 110, and a machine-readable storage medium 120 including the instructions 121-124 for detecting user posture. The computing device 100 may be, for example, a chip set, a desktop computer, a workstation, a notebook computer, a slate computing device, a portable reading device, a wireless email device, a mobile phone, or any other device capable of executing the instructions 121-124. In certain examples, the computing device 100 may be connected to additional devices such as sensors, displays, etc. to implement the method of FIG. 3 below.

The processor 110 may be, at least one central processing unit (CPU), at least one semiconductor-based microprocessor, at least one graphics processing unit (GPU), other hardware devices suitable for retrieval and execution of instructions stored in machine-readable storage medium 120, or combinations thereof. For example, the processor 110 may include multiple cores on a chip, include multiple cores across multiple chips, multiple cores across multiple devices (e.g., if the computing device 100 includes multiple node devices), or combinations thereof. The processor 110 may fetch, decode, and execute instructions 121-124 to implement detection of user posture. As an alternative or in addition to retrieving and executing instructions, the processor 110 may include at least one integrated circuit (IC), other control logic, other electronic circuits, or combinations thereof that include a number of electronic components for performing the functionality of instructions 121-124.

The machine-readable storage medium 120 may be any electronic, magnetic, optical, or other physical storage device that contains or stores executable instructions. Thus, machine-readable storage medium 120 may be, for example, Random Access Memory (RAM), an Electrically Erasable Programmable Read-Only Memory (EEPROM), a storage drive, a Compact Disc Read Only Memory (CD-ROM), and the like. As such, the machine-readable storage medium 120 can be non-transitory. As described in detail below, machine-readable storage medium 120 may be encoded with a series of executable instructions for detecting user posture.

Moreover, the instructions 121-124 when executed by a processor (e.g., via one processing element or multiple processing elements of the processor) can cause the processor to perform processes, such as the method of FIG. 3. For example, the detect instructions 121 may be utilized by the processor 110 to detect posture information related to a user's posture. Examples of the posture information may include information related to the user's position in front of a reference point, distance from the reference point, orientation in front of the reference point, ambient light around the reference point and the like. The reference point may be a sensor, a display, content being displayed, a keyboard, a mouse, and the like.

The posture information may be detected by sensory inputs (not shown) interfacing with the processor 110, such as, a camera sensor, an infrared sensor, a proximity sensor, a weight sensor, and the like. The processor 110 may receive the detected information from the sensory inputs. The store instructions 122 may be utilized by the processor 110 to store the detected information, such as at a database (not shown) and/or the machine-readable storage medium 120. An interval at which the posture information is detected and/or stored may be determined by the instructions 121 and/or 122 and/or set by a vendor and/or user. Similarly, one or more areas of the user's body to detect and/or a threshold amount of movement to occur by the user before the posture information is stored may be determined by the instructions 121 and/or 122 and/or set by a vendor and/or user.

For example, input from the camera sensor along with face recognition instructions included in the detect instructions 121, may be utilized by the processor 110 to identify facial features of the user and anchor markers thereto. Then, the store instructions 122 may be utilized by the processor 110 to track and store a movement of the markers, such as along horizontal and vertical axes. For example, the markers may be anchored to eyes, eyebrows, shoulders, a hair line, a nose, a mouth, a neck, and/or a chin of the user. Embodiments are not limited to using markers. For example, embodiments may use other types of geometric or photometric face recognition algorithms.

In one embodiment, tracking the eyes and/or the markers associated therewith may include detecting at least one of a blink rate, a surface area of the eyes, a distance between the eyes and a height difference between the eyes. If the user is wearing eyeglasses, a type of the eyeglasses may also be detected. Tracking the shoulders and/or the markers associated therewith may include detecting at least one of a distance between the shoulders and a height difference between the shoulders.

While embodiments are described with respect to a single user, embodiments are not limited thereto and may apply to a plurality of users. For example, the face recognition instructions may be used to differentiate between the plurality of users and the store instructions 122 may be utilized by the processor 110 to separately store the posture information of each of the plurality users.

The receive instructions 123 may be utilized by the processor 110 to receive input from the user about a region of the user's body experiencing pain. The user input may be input to the computing device 100 via a user interface (not shown) interfacing with the processor 110, such as, a keyboard, a mouse, a display, a camera, an interactive touch interface and the like. For example, the user interface may allow the user to indicate at least one of a neck, a back, a shoulder, and eyes as the region of the user's body experiencing pain, such as via a window shown on the display. The processor 110 may receive the user input from the user interface. The store instructions 122 may be utilized by the processor 110 to store the received user input.

The provide instructions 124 may be utilized by the processor 110 to provide recommendations for a change to at least one aspect of the user's posture and the user's environment based on the stored information and the received input. For example, the provide instructions 124 may be utilized by the processor 110 to initially analyze the posture information to identify one or more non-neutral positions of the user. A neutral position may be a position in which the user is upright and balanced. The store instructions 124 may, for example, store the coordinates of the markers, when the user indicates and/or the detect instructions 121 determine that the user is in the neutral position The non-neutral position may be a position that deviates from the neutral position, such as when the positions of one or more of the markers deviates by more than a threshold distance compared to that of the neutral position.

Examples of the non-neutral position include a back rounding forward, neck craning, neck flexion, neck extension, neck rotation, torso leaning forward, gaze angle, shoulder abduction, and shoulder extension of the user. In one embodiment, if the user indicates that the neck is experiencing pain, the provide instructions 124 may analyze the stored information related to the neck of the user. For example, the distance between a top of the head or hair and the eyebrows or eyes may be determined. An increasing distance therebetween may indicate increasing neck flexion by the user compared to the neutral position. A decreasing distance therebetween may indicate increasing neck extension by the user compared to the neutral position.

In another example, the distance between the eyes and a tip of the nose may be determined. An increasing distance therebetween may indicate increasing neck flexion by the user compared to the neutral position. A decreasing distance therebetween may indicate increasing by the user neck extension compared to the neutral position. In yet another example, the distance between the chin and a bottom of the neck may be determined. A decreasing distance therebetween may indicate increasing neck flexion by the user compared to the neutral position. An increasing distance therebetween may indicate increasing neck extension by the user compared to the neutral position.

In another embodiment, if the user indicates that the neck and/or back is experiencing pain, the provide instructions 124 may analyze the stored information related to the neck and/or back of the user. For example, the distance between the eyes may be determined. A decreasing distance therebetween may indicate increasing leaning back by the user compared to the neutral position, which may also cause neck flexion. An increasing distance therebetween may indicate increasing learning forward, neck craning forward and/or back rounding by the user compared to the neutral position, which may also cause neck extension.

In yet another embodiment, if the user indicates that the eyes, neck, shoulder and/or back are experiencing pain, the provide instructions 124 may analyze the stored information related to the head tilt, body rotation, and/or shoulder angle of the user. For example, a difference in height between the eyes may be determined. An increasing difference therebetween may indicate increasing neck tilt by the user towards the right or left shoulder.

In another example, a difference in distance between the shoulders may be determined. A decreasing difference therebetween may indicate increasing torso rotation and/or shoulder abduction or extension by the user in the right or left direction. In yet another example, a difference in height between the shoulders may be determined. An increasing difference therebetween may indicate increasing torso tilt by the user towards the right or left side. In still another example, a brightness of the user's environment and/or a difference between the brightness of the user's environment and a display of the user may be determined. An increasing brightness and/or difference in brightness may indicate increasing eye strain to the user.

The machine-readable storage medium 120 may also include filter instructions (not shown) to filter through a plurality of changes to at least one aspect of the user's posture and the user's environment that are possible based on the stored information and the received input, to provide the one or more changes that target only the region of the user's body experiencing the pain. Thus, once the one more causes for the one or more regions of the user's body experiencing pain are determined, the provide instructions 124 may be utilized by the processor 110 to provide a targeted recommendation for a change to at least one aspect of the user's posture and the user's environment. The recommendation may be provided via, for example, a graphic on the display and/or an audible voice of a speaker.

For example, if it is determined that that user's back is rounding forward, the provide instructions 124 may suggest that the user lean back and/or increase a zoom or magnification of characters displayed to provide increased visibility. A magnitude of the suggested zoom may be based on a viewing distance of the user. If it is determined that that user's neck is craning, the provide instructions 124 may suggest that the user adjust a height or depth of the display and/or increase the zoom.

For neck flexion, the provide instructions 124 may suggest the user raise the display to eye level so that the user's head is properly balanced over the shoulders. For neck extension, the provide instructions 124 may suggest the user sit back and lower the display so that the user's head is properly balanced over the shoulders of the user. Further, if the user is wearing multifocal eyeglasses, the user may be able to more easily view the screen through a lower portion of a lens of the multifocal glasses due to the above suggestion.

When the provide instructions 124 determine that there is undue neck rotation, moving the display may be suggested, such as from a side to in front of the user. Also, if the display includes more than one monitor, moving the more frequently used monitor to be directly in front of the user may be suggested. If it determined that the user is leaning such that the user's torso is at an angle, the provide instructions 124 may suggest that the user realign their torso into a non-angled, neutral and supported position.

For shoulder abduction, the user's arm may be extending too far outward and possibly causing the user's back to pinch. Thus, the provide instructions 124 may suggest that the user bring one or more shoulders inward and/or change a hardware arrangement. For instance, the provide instructions 124 may suggest that the user move a mouse inward and/or replace a classic keyboard, which may too wide for the user, with a narrower keyboard, such as a keyboard that lacks a numeric keypad.

For shoulder extension, the user's arm may be stretched too far forward. Thus, the provide instructions 124 may suggest that the user bring one or more shoulders back and/or change a hardware arrangement. For instance, the provide instructions 124 may suggest that the user move a mouse closer in and/or if a touch screen is being used, to move the touch screen in closer and/or point the touch screen at an upward angle so that an elbow of the user is closer and tucked in.

If eye pain or strain is detected, the provide instructions 124 may determine that eyes are too dry and/or that the ambient brightness is insufficient. Therefore, the provide instructions 124 may suggest lowering the display in order to lower a gaze angle of the user, such as from 0 minutes to a range between negative 15 and 35 minutes, like negative 25 minutes. Lowering the gaze angle may cause a greater portion of eyelids of the user to cover the user's eyes, thus providing greater lubrication. The term minute may refer to one sixtieth (1/60) of one degree. If the ambient brightness is determined to be insufficient, such as via the light sensor, the provide instructions 124 may suggest changing the contrast of the display, such as by increasing a contrast ratio.

The provide instructions 124 may also provide more general immediate or non-immediate suggestions. Examples of the immediate suggestions may include suggestions to stand up or move, breathe, blink more, sit back in a comfortable position, vary a seating position, and the like. Examples of the non-immediate suggestions may include suggestions to have the user's eyes checked, such as for new eyeglasses, to find and remove sources of glare, to exercise to reduce stress, and the like. Further, the provide instructions 124 may provide any combination of the above suggestions as well as other types of similar suggestions related to improving the user's posture or environment.

Alternatively, instead of waiting for the user to input the region of the user's body experiencing pain, embodiments may preemptively provide recommendations for a change to at least one aspect of the user's posture and the user's environment, such as via an audio or on-screen reminder for the user to correct their posture based on the stored information.

While embodiments have generally been described with respect to a seated position of the user, embodiments are not limited thereto. For example, the user may be standing, lying down, and the like, such as if the user is using a mobile device and/or a device including a touch interface. For instance, the user may be in various positions while using a tablet.

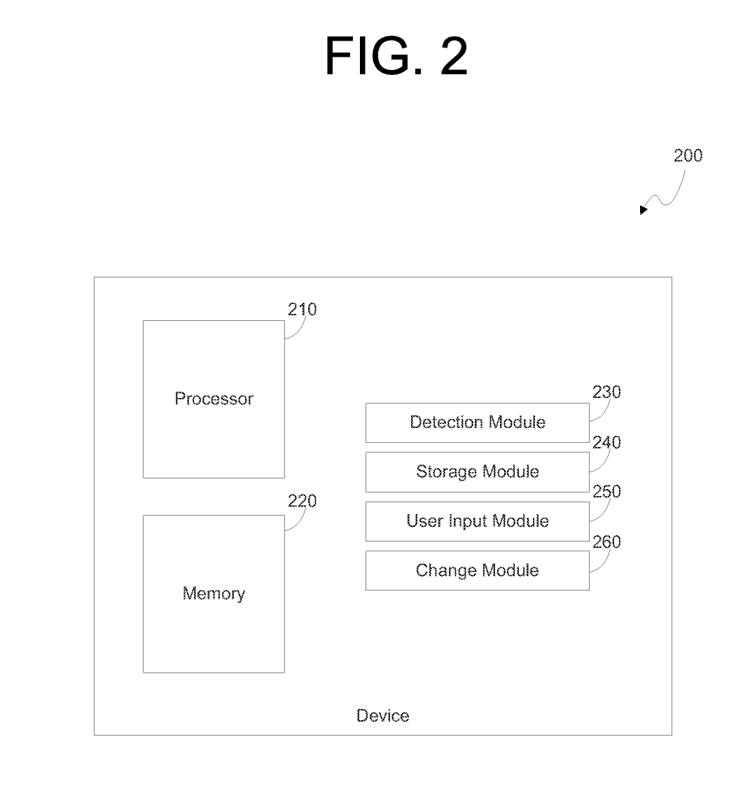

FIG. 2 is an example block diagram of a device to detect user posture. The device 200 may be a desktop computer, a work station, a notebook computer, a slate computing device, a portable reading device, a wireless device, a computing device and the like. In this embodiment, the device 200 includes a processor 210, a memory 220, a detection module 230, a storage module 240, a user input module 250, and a change module 260. The processor 210 may be a CPU, a GPU, or a microprocessor suitable for retrieval and execution of instructions from the memory 220 and/or electronic circuits configured to perform the functionality of any of the modules 230, 240, 250 and 260 described below.

Each of the modules 230, 240, 250 and 260 may include, for example, hardware devices including electronic circuitry for implementing the functionality described below. In addition or as an alternative, each module may be implemented as a series of instructions encoded on a machine-readable storage medium and executable by the processor 210. In embodiments, some of the modules 230, 240, 250 and 260 may be implemented as hardware devices, while other modules are implemented as executable instructions.

The detection module 230 is to detect posture information related to a user's posture, as explained above. A sensor module (not shown) including at least one of a camera, a proximity sensor, a light sensor, an infrared sensor and a weight sensor may detect and transmit the posture information to the detection module 230.

The storage module 240 is to store the detected information, as explained above. For example, the storage module 240 may include a database the store coordinates of a plurality of user markers over a time period, the plurality of user markers to indicate a position of at least one of a facial and body feature of the user based on the detected information.

The user input module 250 is to receive input from the user about a region of the user's body experiencing pain, as explained above. The user input module may include at least one of a microphone, a camera, a keyboard, a mouse and a touch screen to allow the user to indicate at least one of a neck, a back, a shoulder, and eyes as the region of the user's body experiencing pain.

The change module 260 is to provide recommendations for a change to at least one aspect of the user's posture and the user's environment based on the stored information and the received input. A display module (not shown) including at least one of a display and a speaker is to output the recommended change provided by the change module 260. The recommended change to the user's environment may include adjusting at least one of the display used by the user, lighting conditions, and a user interface output on the display. Adjusting the display may include changing at least one of a height, angle and distance of the display with respect to the user. Adjusting the user interface may include changing at least one of a zoom, character height, contrast ratio, and brightness.

In one embodiment, the change module 260 may output notification data to the display module. For example, the notification data may be output to the display as a screen icon, tone, or other reminder that varies to give the user more information on the ergonomic area of concern. For example, if the user is tilting their head to the side and/or complains of neck discomfort, the screen icon may change the neck area of the icon red to indicate the area of concern. Text messages may also be used to notify the user of the recommended changes or corrective actions.

FIG. 3 is an example flowchart of a method 300 for detecting user posture. Although execution of method 300 is described below with reference to the computing device 100, other suitable components for execution of the method 300 can be utilized, such as the device 200. Additionally, the components for executing the method 300 may be spread among multiple devices (e.g., a processing device in communication with input and output devices). In certain scenarios, multiple devices acting in coordination can be considered a single device to perform the method 300. Method 300 may be implemented in the form of executable instructions stored on a machine-readable storage medium, such as storage medium 120, and/or in the form of electronic circuitry.

At block 310, the computing device 100 detects posture information related to a user's posture. The computing device 100 further detects posture information at block 310 related to the user's environment. The detected posture information related to the user's posture may include measuring at least one of a position and angle of at least one of a torso, limb and head of the user. For example, as noted above, the detected information related to a user's posture may include tracking at least one of eyes, eyebrows, shoulders, a hair line, a nose, a mouth, a neck, and a chin of the user. Tracking the eyes may include detecting at least one of a blink rate, a surface area of the eyes, a distance between the eyes, a height difference between the eyes, and a type of eyeglasses of the user. Tracking the shoulders may include detecting at least one of a distance between the shoulders and a height difference between the shoulders. The detected information related to user's environment may include measuring at least one of ambient light, temperature and humidity.

At block 320, the computing device 100 stores the detected information. For example, the computing device 100 may store coordinates of a plurality of user markers over a time period, the plurality of markers to indicate a position of at least one of a facial and body feature of the user based on the detected information. At block 330, the computing device 100 receives input from the user about a region of the user's body experiencing pain, as explained above in further detail above.

At block 340, the computing device 100 provides recommendations for a change to at least one aspect of the user's posture and the user's environment based on the stored information and the received input. For example, the computing device's 100 recommendations for the change to the user's environment may include adjusting at least one of a display used by the user, lighting conditions, and a user interface. Adjusting the display may include changing at least one of a height, angle and distance of the display with respect to the user. Adjusting the user interface may include changing at least one of a zoom, character height, contrast ratio, and brightness.

Further, the computing device 100 may filter through a plurality of possible changes related to at least one aspect of the user's posture and the user's environment that are based on the stored information and the received input in order to only provide the change at block 340 that relates to the region of the user's body experiencing the pain. Thus, the computing device 100 may not provide any changes that do not relate to the region of the user's body experiencing the pain. Also, the computing device 100 provides the change based on analyzing the stored information to determine a trend in the user's posture between the neutral position and the non-neutral position.

Accordingly, embodiments may provide a method and/or device that allows the user to indicate when and where the user is feeling discomfort and that does not interrupt the user. Further, embodiments may store a history or trend of the user's postures over time, which along with the user's indication of where they are experiencing discomfort, may allow embodiments to provide more in-depth and/or targeted recommendations to the user about their posture and/or environment. Thus, embodiments may reduce musculoskeletal and visual discomfort as well as increase user wellness and productivity, in a relatively cost effective and easy to use and/or deployable manner.

1. A method for posture detection, comprising:
detecting posture information related to a user's posture;
storing the detected information;
receiving, during the detection, input from the user if a region of the user's body experiences pain; and
providing a recommendation for a change to at least one aspect of the user's posture and the user's environment based on the stored information and the received input, the recommendation to be provided only if the input from the user is received.

2. The method of claim 1, wherein,
the providing the recommendations for the change to the user's environment includes adjusting at least one of a display used by the user, lighting conditions, and a user interface,
adjusting the display includes changing at least one of a height, angle and distance of the display with respect to the user, and
adjusting the user interface includes changing at least one of a zoom, character height, contrast ratio, and brightness.

3. The method of claim 1, wherein the receiving the input includes the user indicating at least one of a neck, a back, a shoulder, and eyes as the region of the user's body experiencing pain.

4. The method of claim 3, wherein the providing filters through a plurality of changes to at least one aspect of the user's posture and the user's environment that are possible based on the stored information and the received input to provide the change that relates to the region of the user's body experiencing the pain and to not provide the change that relates to a region of the user's body not experiencing the pain.

5. The method of claim 4, wherein,
the providing provides the change based on analyzing the stored information to determine a trend in the user's posture between a neutral position and a non-neutral position, and
the non-neutral position includes at least one of a back rounding forward, neck craning, neck flexion, neck extension, neck rotation, torso leaning forward , gaze angle, shoulder abduction and shoulder extension of the user.

6. The method of claim 1, wherein,
the detecting further includes detecting information related to the user's environment,
the detecting information related to the user's posture includes measuring at least one of a position and angle of at least one of a torso, limb and head of the user, and
the detecting information related to user's environment includes measuring at least one of ambient light, temperature and humidity.

7. The method of claim 6, wherein,
the detecting information related to a user's posture includes tracking at least one of eyes, eyebrows, shoulders, a hair line, a nose, a mouth, a neck, and a chin of the user,
tracking the eyes includes detecting at least one of a blink rate, a surface area of the eyes, a distance between the eyes, a height difference between the eyes, and a type of eyeglasses of the user, and
tracking the shoulders includes detecting at least one of a distance between the shoulders and a height difference between the shoulders.

8. The method of claim 1, wherein the storing stores coordinates of a plurality of user markers over a time period, the plurality of markers to indicate a position of at least one of a facial and body feature of the user based on the detected information.

9. A device comprising:
a detection module to detect posture information related to a user's posture;
a storage module to store the detected information;
a user input module to receive input from the user if a region of the user's body experiences pain, while the posture information is detected; and
a change module to provide a recommendations for a change to at least one aspect of the user's posture and the user's environment based on the stored information and the received input, the recommendation to be provided only if the input from the user is received.

10. The device of claim 9, wherein the storage module includes a database to store the store coordinates of a plurality of user markers over a time period, the plurality of markers to indicate a position of at least one of a facial and body feature of the user based on the detected information.

11. The device of claim 9, wherein the user input module includes at least one of a microphone, a camera, a keyboard, a mouse and a touch screen to allow the user to indicate at least one of a neck, a back, a shoulder, and eyes as the region of the user's body experiencing pain.

12. The device of claim 9, further comprising:
a display module including at least one of a display and a speaker to output the change provided by the change module, wherein
the recommended change to the user's environment includes adjusting at least one of the display used by the user, lighting conditions, and a user interface output on the display,
adjusting the display includes changing at least one of a height, angle and distance of the display with respect to the user, and
adjusting the user interface includes changing at least one of a zoom, character height, contrast ratio, and brightness.

13. The device of claim 9, further comprising:
a sensor module including at least one of a camera, a proximity sensor, a light sensor, an infrared sensor and a weight sensor to measure and transmit information related to a user's posture to the detection module.

14. A non-transitory computer-readable storage medium storing instructions that, if executed by a processor of a device, cause the processor to:
detect posture information related to a user's posture;
store the detected information;
receive input from the user if a region of the user's body experiences pain, while the posture information is detected; and
provide a recommendations for a change to at least one aspect of the user's posture and the user's environment based on the stored information and the received input, the recommendation to be provided only if the input from the user is received.

15. The non-transitory computer-readable storage medium of claim 14, further comprising instructions that, if executed by the processor, cause the processor to:
filter through a plurality of changes to at least one aspect of the user's posture and the user's environment that are possible based on the stored information and the received input to provide the change that targets only the region of the user's body experiencing the pain.
